(19) United States
(12) Patent Application Publication
(10) Pub. No.: US 2004/0106952 A1
(43) Pub. Date: Jun. 3, 2004

(54) Treating arrhythmias by altering properties of tissue

(76) Inventor: Daniel M. Lafontaine, Plymouth, MN (US)

Correspondence Address: STEPHEN R. SCHAEFER, Fish & Richardson P.C., P.A., Suite 3300, 60 South Sixth Street, Minneapolis, MN, 55402, US

(21) Appl. No.: 10/308,816

(22) Filed: Dec. 3, 2002

(51) Int. Cl. A61N 1/36

(52) U.S. Cl. 607/3

(57) ABSTRACT

A device for treating arrhythmia comprising an endoluminal member implantable within a region of a vascular lumen, wherein the member is configured to contact an inner wall of the lumen and alter the properties of tissue proximate the region. Methods of treating arrhythmias using the device are also disclosed.

### TECHNICAL FIELD

The invention relates generally to vascular medical devices and more particularly, to an implantable device and using the device to treat arrhythmias.

### BACKGROUND

Atrial fibrillation, identified as the most common arrhythmia, is a rapid and irregular rhythm in the heart's upper chambers. Atrial fibrillation is known to be the most common risk factor and cause of stroke as well as increased risk of death. The prevalence of atrial fibrillation increases with age, doubling in each decade of age after 50.

Atrial fibrillation has for decades been understood to be maintained by the existence of multiple-reentrant wavelets occurring in random order in the atrium. The source, or sources, of atrial fibrillation have only recently begun to be understood. One source for atrial fibrillation is believed to be rapidly firing focus in or close to the pulmonary veins. Other sources of atrial fibrillation are believed to be similarly triggering foci, located in or near the superior vena cava with cardiac musculature extending from the right atrium, the ligament of Marshall, the insertion site of the vein of Marshall, the crista terminalis, the coronary sinus, and the left atrial posterior free wall.

An approach to curing atrial fibrillation that has been recently studied is to map the foci to determine their location, and then ablate the foci using a catheter ablation technique, for example, the application of local radio frequency (RF) energy. This approach is limited by the difficulties in mapping the foci, a high degree of reoccurrence (because although some foci may be ablated, others may reappear), and a high incidence of stenosis. Another approach that has been studied also uses catheter ablation but creates circumferential lesions around the pulmonary vein ostia. This is done to electrically isolate the triggering foci from the atrium. The success rate for this approach has not been found to be significantly different from the focal ablation approach.

Atrial fibrillation is often associated with heart failure, a condition where a damaged or overworked heart cannot pump blood effectively. In atrial fibrillation, the heart rhythm loses its coordination, and the upper chamber races ineffectively at 300 to 600 beats per minute rather than a normal rate of about 60 to 100 beats per minute. The pumping power of the heart can be cut by a third, thereby increasing the possibility of stagnating at least a portion of the blood. Stagnant blood results in clotting, which may elevate the risk of stroke fivefold.

### SUMMARY OF THE INVENTION

Certain embodiments of the invention provide a device for treating arrhythmia by altering the conductive properties of a region within a patient's vasculature. An implantable device can be used to directly influence the tissues associated with arrhythmia within a region of an ostium of a heart chamber. In a sense, the conductive properties can become altered by “remodeling” the tissue cells that surround a placed or implanted device. The region near the device can therefore be transformed to a non-arrhythmiogenic area, thereby minimizing and/or preventing irregular electrical signals. The trigger points or foci that can initiate arrhythmia (e.g., atrial fibrillation) or conditions for arrhythmia can also be eliminated. The conductive properties of the region near the device can be altered using a variety of mechanisms such as, for example, inducing necrosis on the tissue, eluting a therapeutic drug to the tissue, applying pressure on the tissues, adding mechanical strength to weakened arrhythmiogenic tissue, or combinations thereof.

In an aspect of the invention, a device for implantation in a vascular lumen within an ostium of a heart chamber is provided. The device includes an endoluminal member configured to contact an inner wall of the vascular lumen and alters the properties of tissue near the region at which it is implanted. Upon implantation, the endoluminal member can support the region within the vascular lumen with sufficient strength to prevent stenosis.

In another aspect, a device according to the invention, can include a generally tubular body that enables blood to flow through the tubular body, and has an outer surface shaped substantially complementary with the shape of an inner wall of the ostium to contact the inner wall, an opening at one end of the tubular body having a first outer diameter, and a second outer diameter at a location on the tubular body. The second outer diameter is smaller than the first outer diameter, and larger than the smallest inner diameter portion of the ostium, so that the tubular body is restrained within the vascular lumen.

In yet another aspect, the invention provides a method of treating arrhythmia that includes implanting an endoluminal device within a vascular lumen, and using the implanted device to alter the properties of a region of tissue near the implanted device. For example, the endoluminal device can be used to alter the conductive properties of an area within a vascular lumen. The endoluminal device can be particularly useful in a muscle sleeve or an ostium of a vein, so that a method can be conducted for example, to treat pulmonary vein initiated atrial fibrillation.

In a further aspect of the invention, treating arrhythmia can be achieved by inducing necrosis at a target site near the implanted device. The device can be used to apply heat or cooling to the tissue in order to alter the conduction properties of a region proximate the device. A controlled injury can be produced by increasing or decreasing the temperature of the device sufficient to induce necrosis of the tissue within that region. In response to the injury, a body can grow healthy tissue to replace the injured tissue, and thereby prevent or minimize arrhythmia. The device and thereby the surrounding tissue, can be heated by one or more techniques such as applying electrical energy, using radio frequency, or using an inductive device that self-heats, or combinations thereof. Alternatively, the device can be cooled using a coolant.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic of an embodiment of the device.

FIG. 2 is depiction of a device according to an other embodiment of the invention placed within a vascular lumen (e.g., a pulmonary vein).

FIG. 3 is depiction of a device according a further embodiment of the invention.

Like reference numbers in the figures represent similar elements.

### DETAILED DESCRIPTION OF EMBODIMENTS OF THE INVENTION

The device of the invention is a structure implantable within a region of a patient's vasculature that can transform an arrhythmiogenic area within a vasculature to a non-arrhythmiogenic area by altering the conductive properties of the region. When positioned and implanted in a vascular lumen of an ostium of a heart chamber, the device advantageously initiates a response from the cells and tissue in the region of the device to alter the conductive properties within the vasculature. For example, the trigger points or foci that can initiate arrhythmia (e.g., atrial fibrillation) or conditions for arrhythmia, can be minimized and possibly eliminated. It is presently believed that isolated islands of heart tissue or partially detached portions of tissue can be a cause of arrhythmia. Structurally changing or “remodeling” the cells and thereby the tissue, such as by causing scar tissue, or healthy tissue to form, can reduce the arrhythmiogenic-conducive environment, thereby decreasing and preventing arrhythmia. The modification to the conductive properties can be accomplished in a variety of ways, using the device of the invention. Mechanisms of action to initiate the remodeling include, but are not limited to, mechanical compression, drug-elution, necrosis, adding strength, and combinations thereof.

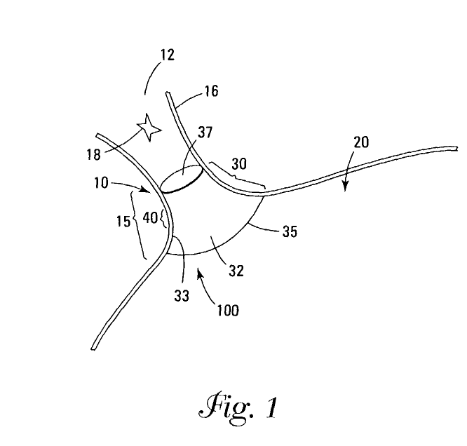

FIG. 1 provides an illustration of an embodiment of the invention, where an exemplary medical device 100 is positioned within an ostium 10 of a vein 16 into a heart chamber 20. Within vein 16, the ostium 10 has a vascular lumen 12 in which device 100 can be implanted and kept in place at a target site 15. A particularly useful target site is the pulmonary sleeve of a pulmonary vein. Another suitable site is a region proximate the junction of a pulmonary vein and the heart tissue of the upper chamber, where the device can provide a bridge between the vein and the chamber.

Arrhythmiogenic tissue can be present within ostium 10. For example, arrythmiogenic trigger points or foci 18 that can initiate disruptive electrical signals to the heart chamber 20 can be present within vascular lumen 20. It is presently believed that disruptive electrical signals (not shown) can emanate from trigger points 18 and cause premature and irregular contraction of a vein 16 and heart chamber 20. This, in turn, can result in the heart's chamber 20 losing its rhythmic coordination relative to the other chambers of the heart.

In the FIG. 1 implementation, device 100 has a generally tubular body 30 having openings 35, 37 on opposite longitudinal ends to allow blood to flow through it. Device 100, when implanted, can be restricted and kept in place within vascular lumen 12 of ostium 10 by virtue of its shape. This can be achieved by shaping the outer surface 32 of body 30 to be substantially complementary and in contact with the inner wall 33 of the ostium. It is contemplated that body 30 can be substantially complementary with inner wall 33 when at least a portion of the circumferential periphery is in contact with a portion of inner wall 33. In one aspect, outer surface 32 of body 30 can conform to the inner wall 33 of the ostium by self-expansion or by assistance of an expansion mechanism, such as an inflatable balloon.

To aid in anchoring the device within the ostium, body 30 can have a tapered design, where one end has a larger outside diameter that is prohibited from entering a smaller diameter lumen. For example, the diameter of end 35 located within heart chamber 20 can be larger than the diameter of a portion 40 of body 30 which is positioned within ostium 10. Device 100 is therefore restricted by having an outer diameter of end 35 larger than that of portion 40, which has the smallest diameter of ostium 10. In an embodiment of the device, the other end 37 located within vein 16 can also, but not necessarily, have a larger diameter than that of portion 40. For ease of maneuverability and patient comfort, body 35 can include a concave arcuate portion.

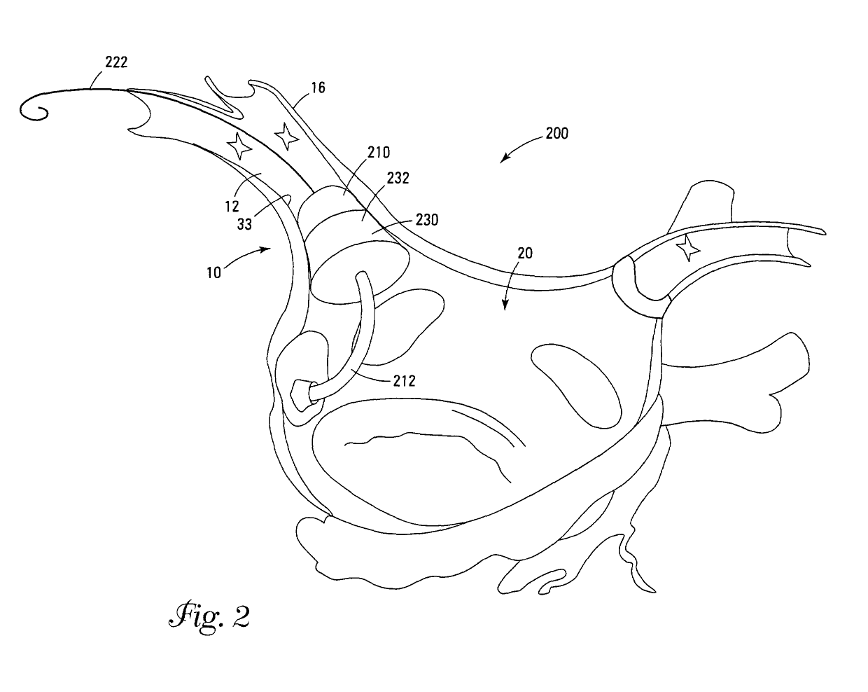

Referring now to FIG. 2, another embodiment of device 200 is shown positioned within vascular lumen 12 of an ostium 10 of a heart chamber 20. Device 200 can include a delivery assembly such as a moveable sleeve, or a catheter 212, as well as an optional guidewire 222. The delivery assembly assists in maneuvering a collapsed tubular body 230 (shown in FIG. 2 as expanded) through a patient and placing it inside vascular lumen 12. An inflatable balloon 210 can be coupled with tubular body 230. Upon positioning a collapsed tubular body 230 at or near a target site 15, balloon 210 can be inflated to an expanded state, thus causing tubular body 230 to expand and fit snugly within ostium 10. The expanded body 230 is thereby anchored, at least in part, to vein 16 and to the heart tissue of chamber 20 to ensure it remains stationary at the target site. In the expanded state, outer surface 232 of body 230 contacts the inner wall 33 of ostium 10. For removal, balloon 210 can be deflated, and guidewire 222, catheter 212 and balloon 210 extracted from the vein.

The device of the invention can be configured such that the structure and physical properties provide optimal strength and pliability. Pliability or flexibility can aid in maneuvering and placing the device within a vascular lumen, while strength of the device can aid in securing the device at a site. As discussed below, strength of the device and the amount of force (e.g., pressure) it can apply to the inner wall of an ostium can also play a role in how the device alters the conductive properties of the region adjacent to an implanted device.

The degree of flexibility (or stiffness) of a device can vary longitudinally along the tubular body to provide more or less support at desired areas. For example, the pliability of tubular body 30 (or 230) can vary along its longitudinal direction such that one end, anchored to the inside walls of chamber 20 is relatively more flexible (i.e., relatively softer or less stiff) than the end located within vein 16. The relatively higher flexibility of the one end located within vein sleeve 16 can allow expansion and contraction of the vein sleeve while maintaining sufficient mechanical anchoring within the pulmonary vein.

A sufficient combination of strength and flexibility can also be accomplished by providing a device having a varying wall thickness. Wall thickness of tubular body 30 (or 230) can vary, particularly along the longitudinal direction of the device. For example, an end 35 of tubular body 30 anchored to the inside walls of the heart chamber 20 can have a relatively thinner wall than the end 37 located within vein 16. In certain applications, such as when the device is implanted in a pulmonary vein sleeve, it may also be desirable to include struts of varying stiffness between the two opposite ends of the device to allow for dynamic longitudinal contraction.

As mentioned above, an endoluminal device of the invention can be self-expanding so that the device enlarges once it is placed within a vascular lumen. A device available commercially from Scimed Life Systems Inc., under the tradename WALL STENT, can be suitable for modification to provide a device for treating arrhythmia. A self-expanding braided device, such as the WALLSTENT, can be modified by braiding wires at varying angles and core diameters to enhance tapered characteristics for frusto-conical ostial placements.

Suitable materials for a device of the invention include, for example, a memory alloy material (e.g., NITINOL), stainless steel, polymers, including but not limited to PTFE or PET, and fabrics such as DACRON™. A mesh material can also be used for the device of the invention.

Treating arrhythmia can be performed according to an embodiment of the invention, by implanting an endoluminal device as described herein, within a vasculature of a body and using the device to alter the conductive properties of the region near or adjacent the device. Methods of the invention treat arrhythmia by directly influencing tissues to transform the arrhythmiogenic area into a non-arrhythmiogenic region. Altering the conductive properties of a region in the vasculature of a body can be accomplished in a variety of ways, including techniques based on mechanical, electrical (e.g., electromechanical or electro-chemical), pharmaceutical, physiological concepts, or combinations thereof.

Certain embodiments according to the invention provide methods that induce necrosis. For example, to circumvent disruptive electrical signals that lead to uncoordinated operation of the heart's chambers, it may be desirable to induce necrosis at a region that has arrhythmiogenic tissue. A controlled injury caused by necrosis can allow a healing response to provide non-arrhythmiogenic new or scar tissue.

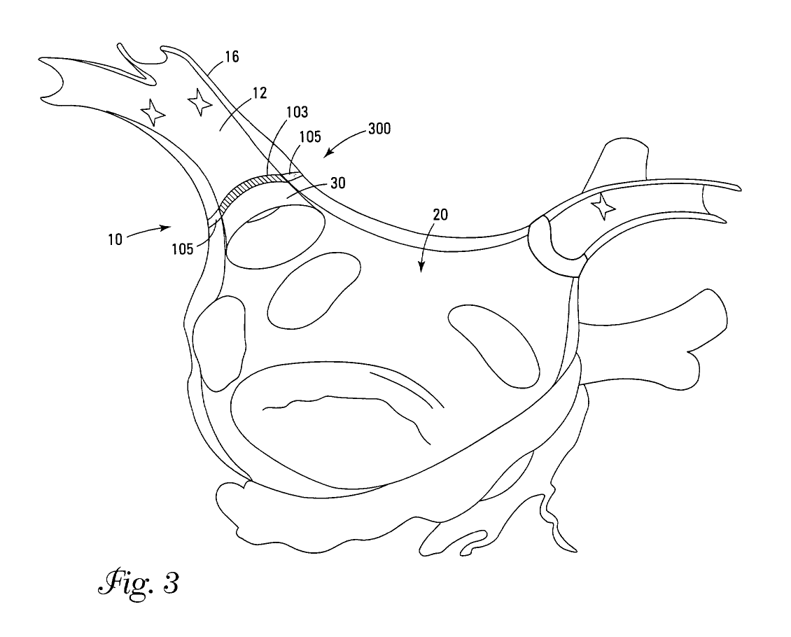

Tissue in the vicinity of a device can be necrotized by, for example, heating or cooling the device, and the surrounding tissue, to a temperature level sufficient to necrotize the region. In an exemplary method, referring now to FIG. 3, a portion 103 of device 300 positioned within a lumen 12 of a vein 16 can be activated so that necrosis can be induced when the temperature of body 30 changes (e.g., heating or cooling). Heat exchange can then occur between device 300 and the surrounding tissue, to change the temperature to a level sufficient for necrotization at or near target site 105. Necrosis of tissue within a heart chamber can be achieved in a similar fashion.

Several methods for heating a device and the surrounding tissue to a temperature sufficiently high for inducing necrosis are available. In one alternative, resistive heating by applying a/c or d/c current to a device having resistive material can be implemented. Another technique can utilize electrical energy by including an electrode and a distal tip on an energy treatment device. The electrode can be located within the patient's body offset proximally of the distal tip. The distal tip can be placed within the patient's body adjacent the tissues. The electrode can then be energized with electrical energy. By electrically contacting the electrode to the tissues with an electrolyte fluid flowing from the electrode to the tissues, necrotization can then occur.

Alternatively, a treatment for arrhythmia can utilize a device energized with RF (radio frequency) energy, where the device includes a material having ferrous or magnetic sections. The target site can be exposed to radio frequency energy to heat the device and thereby the adjacent tissue to consequently induce necrosis. A system for controlled ablation of tissue using RF energy can include a controllable source of RF energy coupled to the device to heat the device and surrounding tissue inductively. The device can include a catheter assembly to provide the energy or heat that can induce necrosis. For example, a catheter assembly can be implemented that includes a distal portion adapted to be inserted into a patient's body, a proximal portion attachable to a source of electrolytic fluid and to a source of RF energy source, and a lumen for delivering fluid from the proximal portion to the distal portion. A porous member can be disposed on the distal portion of the catheter where the porous member defines an interior region in communication with the lumen. An electrode can also be utilized, where the electrode can be disposed in the interior region of the device and configured for coupling to the source of RF energy. RF energy is then transferred from the device to selected tissue areas in a patient's body via electrolytic fluid delivered through the lumen and passing through the porous member. Alternatively, inductive mechanisms for the treating arrhythmia by direct influences on vascular tissue can include devices that use, for example, an internal coil emanating RF (radio frequency) energy, or an external field coil such as an MRI RF (magnetic resonance induction, radio frequency) field.

A further alternative to inducing necrosis and thereby achieving substantially homogeneous non-arrhythmiogenic tissue uses a cooling method. The implantable device and the surrounding tissue can be cooled to a temperature sufficiently low to induce necrosis using, for example, a liquid coolant material transported. For example, the coolant material can be transported through a first lumen of the device to the target site and then exhausted by flowing through a second lumen of the device. Cooling by heat transfer could then occur between the cooled device and surrounding tissue. Coolant material transported through a first lumen can also be allowed to evaporate near the target site. This evaporation step can be performed as a primary technique for cooling to induce necrosis, or it can be performed in addition to the heat transfer technique to provide additional cooling capacity resulting from the heat of evaporation. The coolant material, partially or fully in gaseous form, can then be exhausted through a second lumen. Other ways to achieve controlled necrosis that can be suitable for treating arrhythmia include cryogenic cooling/freezing, ultrasound, microwave, thermal, light or laser applications.

The device, in addition to being capable of altering the conductive properties, can also be used to provide structural support to prevent stenosis of the lumen walls. For example, the stenosis of pulmonary veins can be a complication of RF catheter ablation that could be prevented by heating of an isolation structure (e.g. an endoluminal device) which both ablates by heat necrosis and supports the tissue to prevent negative remodeling, or narrowing of the vascular lumen.

Another method according to the invention can apply the mechanisms of certain angioplasty stenting that induces the mechanisms of restenosis. Such a method can include using the endoluminal device to create an injury, such as with balloon dilation, that will trigger a healing response that promotes the growth of smooth muscle cells, resulting in elimination of the arrhythmiogenic tissue. This remodeling process can create isolation of the arrhythmiogenic foci by creating a homogeneous substrate (e.g. tissue).

Pharmaceutics or drug-based techniques for treating arrhythmia according to a method of the invention can utilize an endoluminal device made from or coated with a material that alters the conductive properties of adjacent tissue. The conductive material can change the conductive properties of the adjacent tissue by, for example, eluting a therapeutically effective amount of a drug(s). Use of optional polymers and proteins can aid in attaching the drugs to the tissue. Alternatively, the body of the device can include reservoirs or porous wells that can hold and deliver the eluting drugs. Suitable conductive materials that can be used to make or coat a device include, but are not limited, to beta blockers, sodium blockers, potassium blockers, hydrogels, tissue growth enhancers, etc. (or combinations thereof). The device can also be manufactured from and/or coated with effective amounts of material that can prevent undesired biological or physiological effects. For example, the device can include a material for preventing restenosis and/or apoptosis. Alternatively, use of a non-conductive material to make or coat a device for treating arrhythmia can “block” a disruptive electrical signal that can trigger arrhythmia.

In an alternative method of the invention, a mechanical-based technique can be utilized. According to one method, an expandable endoluminal device can be used to contact and compress the inner walls of a vascular lumen. By implanting a sufficiently strong device snugly within a vascular lumen, the device can press against the lumen's inner walls and apply sufficient compression (e.g., force) to the tissues. This compression can alter the conductive properties of the region near the device and transform it into a non-arrhythmiogenic area. Advantageously, the endoluminal device can substantially simultaneously provide strength to what may be a deteriorating target site such as a damaged pulmonary sleeve.

Optionally, a plurality of devices can be placed at several different target sites. In some applications it may be desirable to inter-connect one or more devices, to create a complete paving of multiple pulmonary vein orifices, such as common antrum veins. The devices can become connected electrically or physiologically (e.g., tissue bridges) by the remodeling process associated with the implanting of the devices. Whether the mechanism of action for the treatment is necrosis, of by elution of a growth enhancing material or anti-arrhythmic drug, resulting regions of overlap may double dose the eluting material or mechanism of action. However, achieving the consistent connected and substantially homogeneous tissue can still be result, as the arrhythmiogenic region is still transformed to be non-arrhythmiogenic.

It is contemplated that implantable devices to alter arrhythmia can also be effective within the heart chamber itself. For example, an implantable device which substantially completely surrounds a valve annulus could effectively electrically isolated the entire valve annulus by a localized action from the device. This can be accomplished by necrosis or by drug delivery or elution. In another embodiment, a device can be used to electrically bisect a heart chamber by creating a large loop structure that is implanted within the heart chamber to achieve localized anti-arrhythmic action.

Delivery and placement of the device within a vascular lumen can be performed using any known technique that advances a device into a vascular system. One example, as described above, can utilize a guide wire, a catheter, or both, to position the device. Optical aids or components can optionally be coupled with a delivery apparatus to provide visual assistance in placing the device at the desired target site.

A number of embodiments of the invention have been described. Nevertheless, it will be understood that various modifications may be made without departing from the spirit and scope of the invention. Accordingly, other embodiments are within the scope of the following claims

What is claimed is:

1. A device for treating arrhythmia comprising:
an endoluminal member implantable within a region of a vascular lumen, said member configured to contact an inner wall of said vascular lumen and alter the properties of tissue proximate said region.

2. The device of claim 1 wherein said endoluminal member, when implanted, supports said region with sufficient strength to prevent stenoses.

3. The device of claim 1, further comprising a delivery assembly coupled with said endoluminal member.

4. The device of claim 1, further comprising an ablation element formed integrally with said endoluminal member.

5. A medical device comprising
a generally tubular body sized for implantation within an ostium of a vascular lumen into a heart chamber, said tubular body, when implanted within the ostium, enables blood to flow through the tubular body, and said tubular body having
an outer surface shaped substantially complementary with the shape of an inner wall of the ostium to contact an inner wall of the ostium;
a first outer diameter at one opening of said tubular body; and
a second outer diameter at a location on the tubular body, wherein the second outer diameter is smaller than the first outer diameter, and larger than the smallest inner diameter portion of the ostium, so that the tubular body is restrained within the vascular lumen.

6. The medical device of claim 5, wherein the vascular lumen is a pulmonary vein.

7. The medical device of claim 5, wherein the outer surface of the tubular body comprises a concave arcuate portion.

8. The medical device of claim 5, wherein the outer surface of the tubular body comprises a mesh material.

9. The medical device of claim 5, wherein at least a portion of the outer surface of said tubular body comprises an outer coating comprising a substance that alters the conductive properties of adjacent tissue.

10. The medical device of claim 9, wherein said substance is selected from a group consisting of a tissue growth enhancer, a conductive hydrogel, a beta blocking drug, a sodium blocking drug, potassium blocking drug, and combinations thereof.

11. The medical device of claim 5, wherein the generally tubular body comprises a heat conducting material that raises the temperature of tissue proximate the outer surface of the generally tubular body.

12. The medical device of claim 5, wherein said outer surface becomes heated upon application of radio frequency energy in the vicinity of said tubular body.

13. The medical device of claim 5, further comprising a fastening mechanism attached to said tubular body for securing said device in a desired position within the ostium.

14. The medical device of claim 5, wherein said tubular body supports said tissue of said vascular lumen with sufficient support to prevent stenoses.

15. The medical device of claim 5, wherein said tubular body further comprises a vascular graft.

16. The medical device of claim 5, further comprising an implantation system coupled with said tubular body.

17. The medical device of claim 5, wherein said tubular body is self-expanding.

18. The medical device of claim 5, wherein said tubular body further comprises an anti-stenosis component.

19. The medical device of claim 5, wherein at least a portion of said device has varying longitudinal flexibility.

20. The medical device of claim 5, further comprising a delivery assembly connectable to said tubular body.

21. The medical device of claim 5, further comprising an inflatable balloon sized to expand said tubular body within said vascular lumen.

22. The medical device of claim 20, wherein said delivery assembly comprises a guidewire.

23. The medical device of claim 20, wherein said delivery assembly comprises a catheter.

24. A method of treating arrhythmia, comprising
implanting an endoluminal device in a region within a vascular lumen of a vasculature, wherein said device contacts an inner wall of said vasculature and has a lumen extending through said device;
using said implanted device to alter properties of vascular tissue proximate said implanted device.

25. The method according to claim 24, further comprising using said implanted device to alter the conductive properties of said vascular tissue.

26. The method according to claim 24, further comprising inducing necrosis on a portion of said tissue proximate said device.

27. The method according to claim 26, wherein necrosis is induced by altering the temperature of said device.

28. The method according to claim 26, wherein necrosis is induced by applying electrical current to said device.

29. The method according to claim 26, wherein necrosis is induced by using said device to expose said tissue to a coolant material.

30. The method according to claim 24, wherein said device comprises an electrode element and the method further comprises
transmitting radio frequency energy to said electrode; and
allowing said radio frequency energy to transfer from said device to said tissue.

31. The method according to claim 24, further comprising attaching said device to said inner wall of said vasculature.

32. The method according to claim 24, wherein using said implanted device applies sufficient pressure on said tissue to alter the conductive properties of said tissue.

33. The method according to claim 24, wherein said device comprises a substance comprising a therapeutic drug; and said method further comprises allowing said drug to elute onto said tissue.

34. The method according to claim 24, wherein said therapeutic drug comprises a material selected from a group consisting of a tissue growth enhancer, a conductive hydrogel, a beta blocking drug, a sodium blocking drug, potassium blocking drug, and combinations thereof.

35. The method according to claim 24, wherein said vascular lumen is within a pulmonary vein.

36. The method according to claim 24, wherein said region is at an ostium of a heart chamber.
